(19) United States
(12) Patent Application Publication
(10) Pub. No.: US 2012/0232533 A1
(43) Pub. Date: Sep. 13, 2012

(54) DEVICE AND METHOD FOR TENSIONING AN ELONGATE MEMBER

(75) Inventors: Michael Scott Veldman, Memphis, TN (US); Jeffrey Warren Beale, Bartlett, TN (US); Robert H. Humphries, Jr., Danbury, CT (US); Hanspeter Robert Bayer, Meriden, CT (US)

(73) Assignee: SONOMA ORTHOPEDIC PRODUCTS, INC., Santa Rosa, CA (US)

(21) Appl. No.: 13/508,519

(22) PCT Filed: Dec. 1, 2010
(86) PCT No.: PCT/US10/58553

§ 371 Date: May 7, 2012

Related U.S. Application Data
(60) Provisional application No. 61/266,946, filed on Dec. 4, 2009.

(51) Int. Cl. A61B 17/00

(52) U.S. Cl. 606/1

(57) ABSTRACT

An elongate member having a fixed end is passed through a plurality of grippers that impart tension in the elongate member and prevent the tension from being released. At least one gripper is movable while at least one other can be fixed. The movable gripper travels along the elongate member toward the fixed end then reverses direction, grips the elongate member, and carries the elongate member away from the fixed end, thereby creating tension in the elongate member. At least one gripper prevents the tension from being released. In some embodiments, the elongate member passes through a rotatable shaft with a lumen. The shaft can be in communication with a crank, whereby operation of the crank rotates the shaft.

### BACKGROUND

1. Field of the Invention

Embodiments of the present invention relate to tensioning a flexible elongate member, such as a wire, cable, or tether. Some embodiments relate to the use of an elongate member in surgical applications, such as use in a segmented intramedullary or IM nail, which is used in the treatment of long bone fractures.

2. Description of the Related Art

The use of elongate members is well known in the art. Elongate members are used to conduct electrical current, support bridges and buildings, and carry data worldwide virtually instantaneously. An elongate member can be any flexible elongated member capable of maintaining tension up to its yield strength, such as but not limited to, wire, cable, tether, rod, threaded rod, rope, line, chain, twine, yarn, and string. An elongate member can be a variety of materials, such as but not limited to, copper, steel, iron, tin, silver, gold, cotton, hemp, sisal, nylon, aramids, polyester, and polypropylene. Depending on the application, an elongate member can be monofilament or multifilament, stranded or unstranded, coated or uncoated, sheathed or unsheathed.

Elongate members are generally tensioned in order to carry a load. Conventional elongate member tensioners typically employ a ratchet mechanism to impart the tensile force on the elongate member and prevent the elongate member from slipping as the tension increases. For instance, U.S. Pat. No. 2,980,974 (Santis), discloses an elongate member tensioner with a ratcheting mechanism. However, by the nature of the ratchet, such tensioners have adjustment that is limited to the spacing of the ratchet teeth. Thus, such tensioners generally cannot maintain levels of tension that do not correspond with the location of the ratchet teeth.

The elongate member is normally secured after it is tensioned. This is usually achieved using a crimp, clamp, or compression device. Typically, such securing devices require the use of unique tools, such as special pliers, which are separate from the tensioning device. Thus, a person installing the elongate member can be required to provide, maintain, and manipulate separate devices to tension and lock the elongate member.

A surgical elongate member is a special type of elongate member that is used in a variety of medical procedures, such as fracture fixation, total hip arthroplasty, and other trauma surgery. A surgical elongate member is typically a slender length of surgical elongate member manufactured from a biocompatible material, such as but not limited to, titanium, stainless steel, or tungsten. Surgical elongate member can be used to provide stability and support to a fractured or otherwise injured area, or to affix other objects to the bone. Often, the surgical elongate member must be tensioned to secure it and provide adequate stability and support. If the tension in the surgical elongate member is too slight, the surgical elongate member can shift from the injured area and fail to provide the stability and support desired. On the other hand, if the surgical elongate member is too highly tensioned, it can cause injury to the patient's body or other implanted devices.

Like other types of tensioners discussed above, surgical elongate member tensioners often employ a ratchet. For instance, U.S. Pat. No. 5,312,410 (Miller et al.) discloses an elongate member tensioner with a ratcheting mechanism. As stated, such mechanisms have adjustment limited to the ratchet tooth spacing.

### SUMMARY

A tensioning device that provides continuous and uninterrupted adjustment could provide improved control of tension imparted to an elongate member. Additionally, a tensioning device with the ability to secure an elongate member would remove the need for separate tools, enhance safety, and simplify the installation process. To those ends, the disclosed inventions generally seek to provide full tension control of an elongate member and the independent ability to secure an elongate member under tension.

In accordance with some embodiments disclosed herein, a tensioning device and method for tensioning are provided. The device can comprise a body with proximal and distal ends. Some embodiments include a handle to facilitate handling and control of the device. In some embodiments, the device and method may be for compressing a device or bone by tensioning an element connected to the device or bone. In various non-limiting embodiments, the device can be called a tensioner, a compressor, a gun, a tensioner gun instrument, a cable gun tensioner, or other names. In one embodiment the cable gun tensioner instrument consists of two unique and independent mechanisms. The first mechanism is a dual piston mechanism that can be cycled by pulling the trigger of the tensioner. The first piston cycles back and forth thereby pulling the cable through the tensioner while the second piston serves to hold the tension applied to the cable by the first piston. The entire two piston mechanism abuts a spring that is intended to indicate the tension being applied to the cable.

Some embodiments comprise a plurality of grippers. In one embodiment, the grippers are aligned to permit an elongate member with proximate and distal ends to pass unimpeded in one direction. In other embodiments, the grippers are not aligned. In one embodiment, the grippers permit the elongate member to freely move in the proximate direction, but prevent the elongate member from moving in the distal direction. Thus, when the distal end of the elongate member is fixed, the grippers can impart tension to the elongate member and prevent such tension from being released.

In some embodiments, the grippers work in conjunction. One or more grippers can traverse a length of the elongate member and one or more grippers can be fixed. Upon reaching a desired length of travel in the distal direction, the movable gripper reverses course and begins moving proximally. In doing so, the movable gripper locks onto the elongate member, thereby carrying the elongate member proximally and increasing the tension in the elongate member. Upon reaching a desired proximal length of travel, the movable gripper begins travelling distally again and releases its hold on the elongate member. When the movable gripper releases, the fixed gripper locks the elongate member, thus preventing the elongate member from moving in the distal direction and the tension from being released. Some embodiments can have a mechanism to release the movable gripper's grip on the elongate member. Various embodiments are configured to measure the amount of tension in the elongate member. For instance, one embodiment comprises a spring coupled to an indicator to reflect the amount of tension in the elongate member.

Certain embodiments employ a trigger connected to one or more of the grippers by a linkage. In one embodiment, the linkage is designed such that when the trigger is moved proximally, the movable gripper also moves distally. In another embodiment, moving the trigger proximally moves the gripper proximally. Other embodiments have alternate linkage configurations.

Embodiments of the device can also comprise a rotatable shaft with a lumen. Various embodiments align the plurality of grippers so that the elongate member can simultaneously pass through the grippers and the lumen. However, this is not required. In some embodiments, the lumen is configured to interface a compressible implant, thereby locking the elongate member in place. In some embodiments, the shaft can be controlled by a crank. In one embodiment, the crank is located at the proximal end of the tensioner and the shaft at the distal end of the tensioner. In some embodiments sets of gears connect the crank to the shaft via an offset rod.

Other embodiments include a method of imparting tension to an elongate member using the plurality of grippers discussed above.

### BRIEF DESCRIPTION OF THE DRAWINGS

These and other features, embodiments, and advantages of the present invention will now be described in connection with preferred embodiments of the invention, in reference to the accompanying drawings. The illustrated embodiments, however, are merely examples and are not intended to limit the invention.

FIG. 1 is a perspective view of a tensioner, according to a first embodiment of the disclosure.

FIG. 2 is a perspective sectional view of the tensioner shown in FIG. 1.

FIG. 3 is a plan sectional view of the tensioner shown in FIG. 1.

FIG. 4 is a partial plan sectional view of the tensioner shown in FIG. 1 in the extended position.

FIG. 5 is a partial plan sectional view of the tensioner shown in FIG. 1 in the retracted position.

FIG. 6 and FIGS. 6A-6D are a schematic chart of an embodiment of a method for imparting tension in an elongate member using a plurality of grippers.

Throughout the figures, the same reference numerals and characters, unless otherwise stated, are used to denote like features, elements, components or portions of the illustrated embodiments. In certain instances, similar names can be used to describe similar components with different reference numerals which have certain common or similar features. In various embodiments, multiples of the same embodiment, or different embodiments of a designated reference number can be used with a prime (′) symbol on the reference number. For example, if a first part with reference number 000 is designated, multiples of the same or a different embodiment of that reference can include a second 000′, third 000″, etc. Moreover, while embodiments of the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION

In accordance with the present disclosure, various embodiments of a device and method for tensioning an elongate member are provided. An elongate member can be any flexible elongated member capable of maintaining tension up to its yield strength, such as but is not limited to, wire, cable, tether, rod, threaded rod, rope, line, chain, twine, yarn, and string. An elongate member can be a variety of materials, such as but not limited to, copper, steel, iron, tin, silver, gold, cotton, hemp, sisal, nylon, aramids, polyester, and polypropylene. Depending on the application, an elongate member can be monofilament or multifilament, stranded or unstranded, coated or uncoated, sheathed or unsheathed. Use of terms related to embodiments of elongate members can be used interchangeably and should be understood to refer to the various types of embodiments of the elongate member being used. For example, cable can be used to describe any number of types of elongate members, but should not be necessarily limited to use only with a cable.

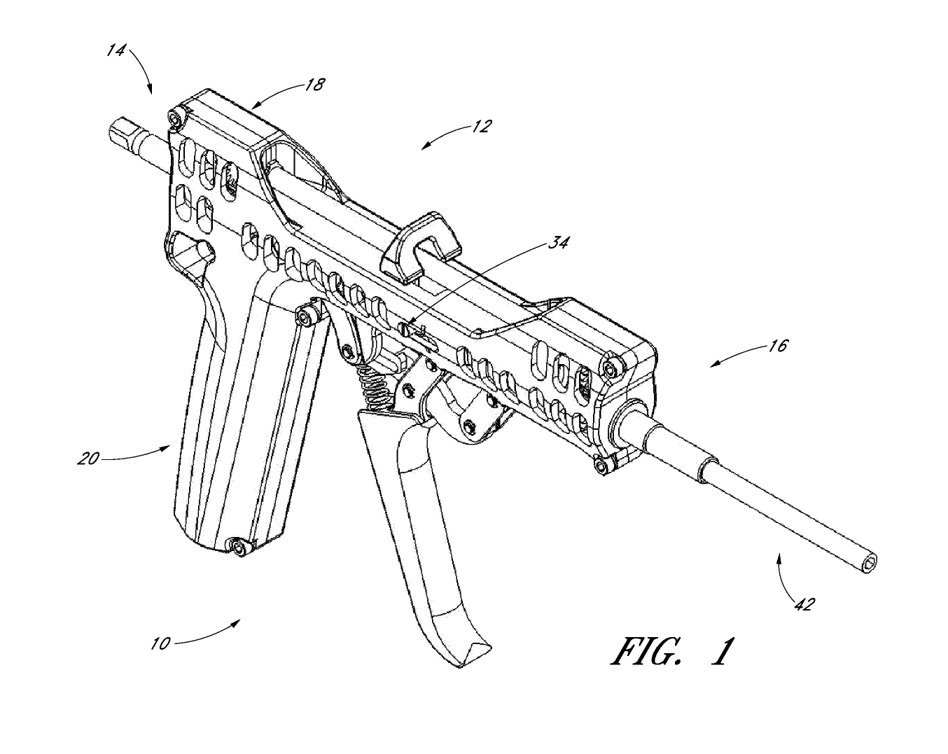

FIG. 1 shows a first embodiment of a tensioning device 10. The illustrated embodiment of the tensioning device 10 comprises a body 12 having a proximal end 14 with a rotatable crank 40, and a distal end 16 with a rotatable shaft 42. The illustrated embodiment of the device 10 has a length of about 320 mm. Various embodiments have other lengths, such as about 100 mm to about 600 mm, about 200 mm to about 500 mm, and about 300 mm to about 400 mm. However, other lengths of the device 10 can be used.

In one embodiment the device 10 can have one or more protective covers 18 connected to the body 12. Embodiments can include a handle 20 interfacing the body 12 or the protective cover or covers 18. The body 12, protective cover or covers 18, and handle 20 can each be metal, plastic, fiberglass, hard rubber, resin, or similar material.

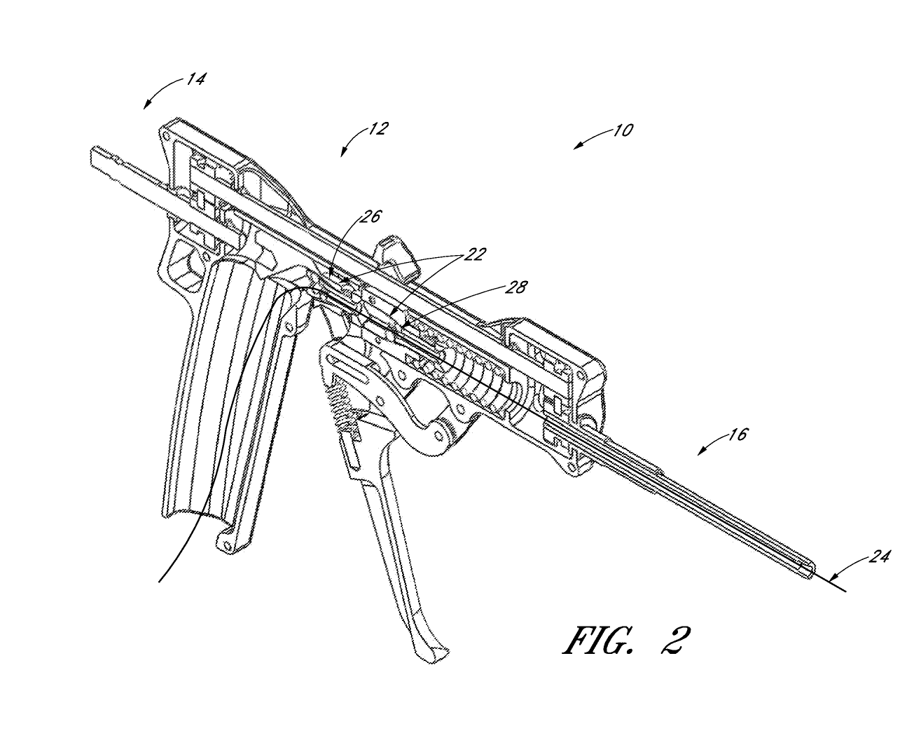

As shown in one embodiment in FIG. 2, the body 12 houses a plurality of grippers 22. The grippers 22 are configured to permit an elongate member 24 to freely move proximally, but not distally. Thus, when the elongate member 24 is distally fixed, the grippers 22 can proximally move the elongate member 24 to impart tension and prevent such tension from being released. In the illustrated embodiment, the grippers 22 are aligned to permit the elongate member 24 to concurrently pass through both grippers 22. In other embodiments, the grippers 22 are not so aligned.

In the illustrated embodiment, the grippers 22 provided are according to U.S. Patent Application No. US 2007/0171540 (Veldman et al.), the disclosure of which is hereby incorporated by reference. In sum, each of the grippers 22 includes one or more spheres held within a hollow conical chamber that tapers from a larger cross section proximally to a smaller cross section distally. The elongate member 24 enters the conical chamber distally, passes between the spheres, and continues out through an axial opening in the proximal end of the chamber. Proximal movement of the elongate member 24 can be freely accomplished, since the spheres tend to advance into the larger diameter of the chamber. Conversely, distal movement of the elongate member 24 tends to draw the spheres into the smaller diameter of the chamber. The tapered walls of the chamber wedge the spheres inwardly against the elongate member, thus preventing distal movement.

It should be noted that other embodiments of the device 10 can employ alternate designs and/or configurations of the grippers 22, such as, but without limitation, wedges, clamps, clasps, springs, fasteners, ratchets, magnets, threads, and the like.

The illustrated embodiment includes a first gripper 26 and a second gripper 28. In some embodiments, in relation to the device body 12, the first gripper 26 remains substantially fixed, while the second gripper 28 travels proximally and distally. In other embodiments, in relation to the device body 12, the first gripper 26 travels proximally and distally and the second gripper 28 remains substantially fixed. In still other embodiments, both grippers 22 can travel proximally and distally. Yet further embodiments include both grippers 22 that are substantially fixed.

In the embodiment shown, the proximal length of travel of the second gripper 28 can be about 3 cm-30 cm. However, other embodiments can have different lengths of travel. For example, an alternate embodiment has a second gripper 28 that travels about 5 cm-20 cm. Another embodiment includes a first gripper that travels about 1 cm-20 cm. Other lengths of travel are possible.

Note that, from the perspective of the grippers 22, proximal movement of the grippers 22 along the elongate member 24 is equivalent to distal movement of the elongate member 24 along the grippers 22. Thus, the grippers 22 will lock to the elongate member 24 when either the elongate member 24 moves distally to the grippers, or the grippers 22 move proximally to the elongate member 24.

Accordingly, when an elongate member 24 is passed through the grippers 22, the second gripper 28 can freely distally traverse a length of the elongate member 24. But when the second gripper 28 travels proximally, the second gripper 28 locks to the elongate member 24. Thus, the second gripper 28 can proximally carry the elongate member 24, thereby increasing the tension in the elongate member 24. During the distal movement of the second gripper 28, tension in the elongate member tends to lock the first gripper 26 to the elongate member 24, thereby impeding the elongate member 24 from moving distally.

Depending on the application, alternate embodiments of the device 10 can produce various amounts of tension in the elongate 24. For example, but without limitation, the embodiment shown is configured to impart a tension of up to about 100-1,000 pounds into the elongate member 24. However, other embodiments of the device 10 are designed to impart other amounts of tension.

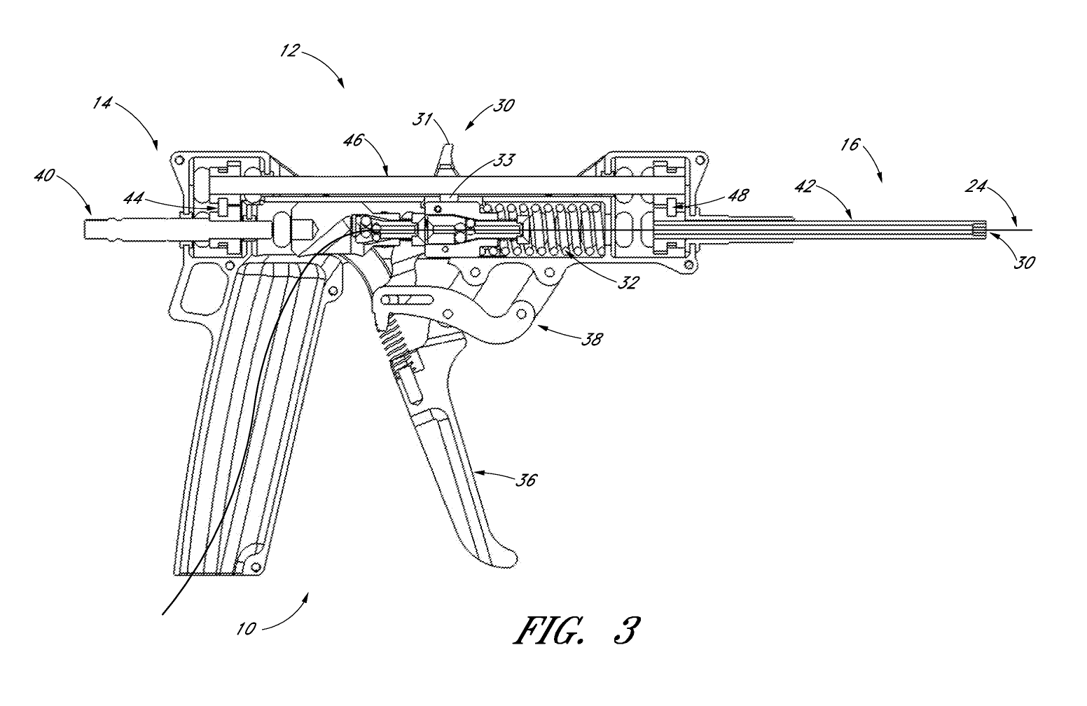

Turning to FIG. 3, some embodiments includes a release mechanism 30 to release the lock of grippers 22 on the elongate member 24. In the illustrated embodiment, the release mechanism 30 is comprised of a release knob 31 connected to a release linkage 33, which is coupled to the second gripper 28. Proximal movement of the release knob 31 interfaces features on the body of the grippers 22, thereby proximally moving the grippers 22 and thus releasing the lock of the grippers 22 on the elongate member 24. Other embodiments use other release mechanism 30 configurations. Some embodiments include a safety feature, such as a detent, to prevent inadvertent operation of the release mechanism 30.

The illustrated embodiment includes a trigger 36 connected to the grippers 22 by a linkage 38. In other embodiments, the trigger 36 is connected to only one of the first gripper 26 or second gripper 28. As shown, the trigger 36 is manually actuated by the hand of the device 10 user. Various other embodiments include a trigger 36 with actuation assistance, such as electrical, hydraulic, pneumatic, or similar actuation.

The linkage 38 can be configured for various manners of operation. For instance, in one embodiment, when the trigger 36 is moved proximally the second gripper 28 is moved distally. In another embodiment, moving the trigger 36 proximally moves the second gripper 28 proximally. Various other embodiments have alternate linkage 38 configurations.

The illustrated embodiment includes a spring 32 and indicator 34 (not shown) for measuring the amount of tension in the elongate member 24. The spring 32 can be, without limitation, a coil spring, wave spring, belleville washer, wave washer, leaf spring, torsion bar, gas spring, or the like. Some embodiments incorporate a sensor that outputs a signal in relation to the force on the spring 32 or in relation to the tension in the elongate 24 directly. The indicator 34 can be, but is not limited to, a mechanical, pneumatic, or electrical gage or the like. The indicator 34 can also be a visual or aural signal configured to activate at a determined level of tension.

As shown, when the second gripper 28 locks to the elongate member 24, the tension of the elongate member 24 is transferred through the second gripper 28 to the spring 32, thereby putting the second gripper 28 in communication with the spring 32 and compressing the same. The spring 32 is connected to the indicator 34, which can be calibrated to measure the amount of force on the spring 32 and thereby register the amount of tension in the elongate member 24. It is to be understood that this is merely one approach for assessing and indicating the force on the elongate member 24, and that other embodiments employing various other measurement and indication means are contemplated.

With continued reference to FIG. 3, the illustrated embodiment of the tensioning device 10 has a rotatable crank 40 at the proximal end 14 and a rotatable shaft 42 at the distal end 16. The crank 40 is in communication with a first set of gears 44, while the shaft 42 is in communication with a second set of gears 48. Both the first set of gears 44 and second set of gears 48 are in communication with an offset rod 46. Thus, rotating the crank 40 transmits the rotational movement to the shaft 42 via the first set of gears 44, the offset rod 46, and the second set of gears 48. The configuration of the sets of gears 44, 48 can be, but is not limited to, spur, helical, beveled, worm, rack and pinion, friction, belt-drive, or the like. The material of the sets of gears 44, 48 can be metal, plastic, or the like.

Alternate embodiments include other approaches for rotating the shaft 42. One embodiment includes a signaling device, such as a button, toggle, or similar, in electronic communication with a motor in rotatable communication with the shaft 42, such that operation of the signaling device initiates the motor to rotate the shaft 42. In another embodiment, the crank is located at the distal end 14 of the device 10 and is in communication with the shaft 42 via gears or the like. Still further embodiments include handles, knobs, grips, or texture features on the shaft 42 to facilitate its rotation.

In the embodiment shown, the rotatable shaft 42 includes a lumen 50 and is connected to the distal end of the body 12 of the tensioning device 10. The shaft 42 can be made from a biocompatible material, such as titanium, stainless steel, or other materials. As shown, the shaft 42 and grippers 22 are aligned so that the elongate member 24 can simultaneously pass through the grippers 22 and the lumen 50. However, this is not required. In one embodiment, the lumen 50 is configured to interface a compressible implant 52 (not shown), for securing the elongate member 24 under tension. For example, in one embodiment the lumen 50 is configured to interface a threaded collet as disclosed in U.S. Pat. No. 7,172,595 (Goble). Other embodiments include alternate configurations of the lumen 50.

In the illustrated embodiment, the movement of the trigger 36, grippers 22, spring 32, and indicator 34 are independent of the movement of the crank 40, sets of gears 44, 48, offset rod 46, shaft 42, and lumen 50. Accordingly, the ability to tension the elongate member 24 is independent of the ability to secure the elongate member 24. In other embodiments, these abilities can be interrelated, such as by a linkage, belt, spring, cam, gearing, or similar.

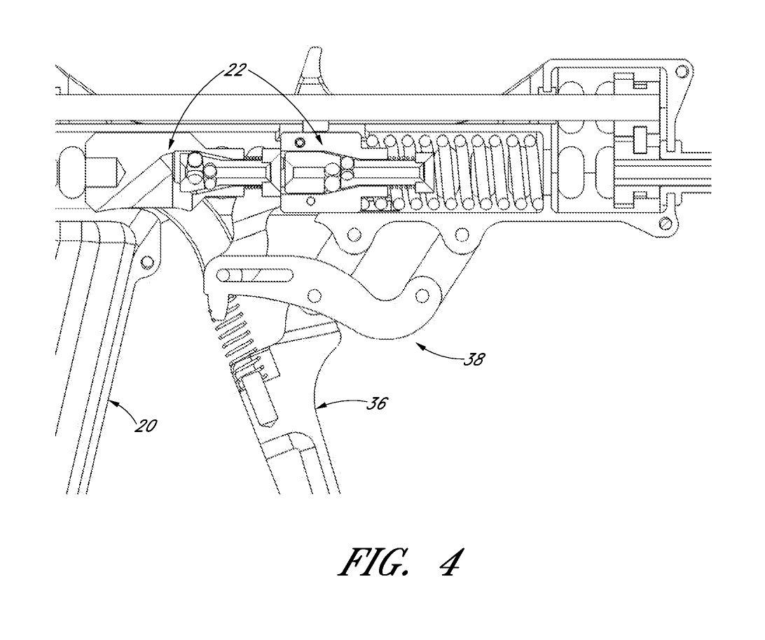

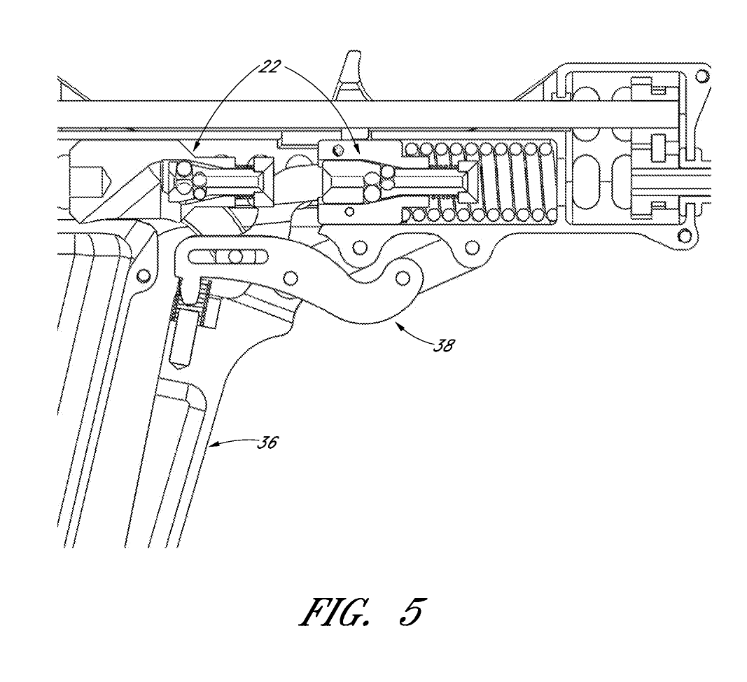

FIGS. 4 and 5 illustrate the internal operation of one embodiment of the device 10. In FIG. 4, the device 10 is shown in the extended state, in which the trigger 36 is a distance apart from the handle 20 and the linkage 38 is distally disposed. As the trigger 36 is retracted toward the handle 20, the linkage 38 moves proximally. This retracted state is illustrated in FIG. 5. Due to the configuration of the linkage 38 and its connection to the grippers 22, the proximal travel of the linkage 38 distally encourages the grippers 22. When the trigger 36 is allowed to return to its initial extended position, the grippers 22 also return to their initial position, thereby allowing the trigger 36 to be retracted again. Note that FIGS. 4 and 5 are only one embodiment and that other configurations are contemplated.

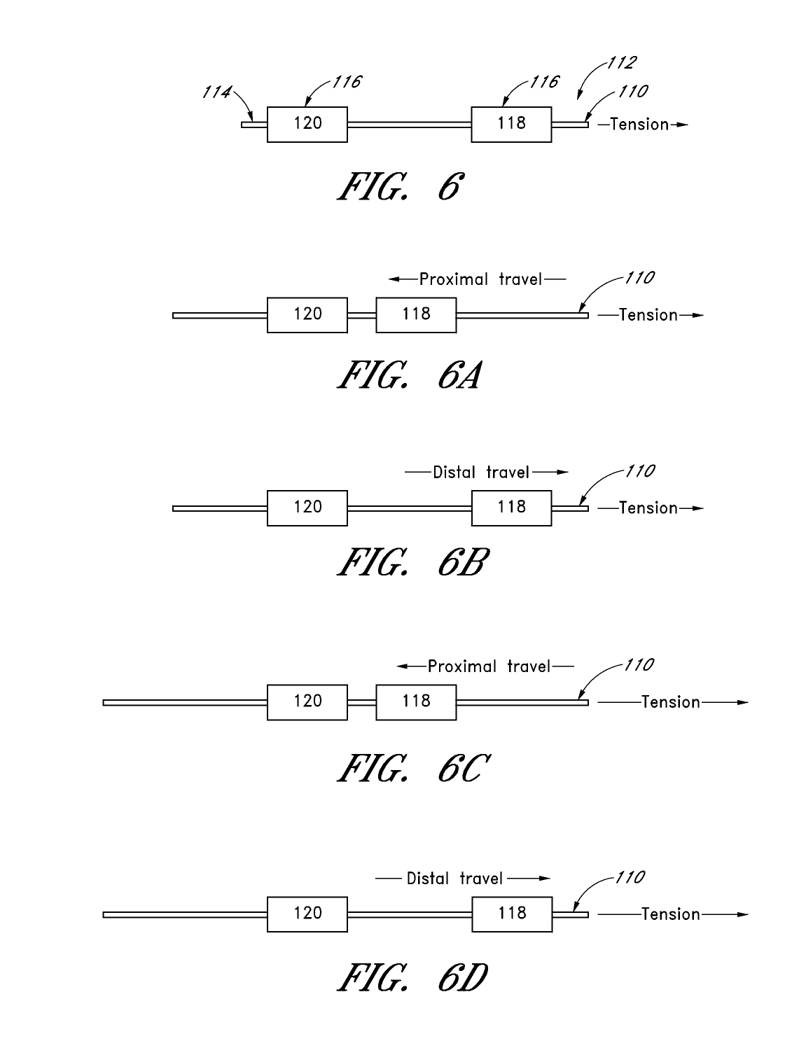

FIG. 6 is a schematic chart illustrating an embodiment of a method for tensioning an elongate member using a plurality of grippers. In the illustrated embodiment, a plurality of grippers 116 are provided along with an elongate member 110 with a fixed distal end 112 and a free proximal end 114. The grippers 116 are designed as to allow proximal movement of the elongate member 110 and prevent distal movement of the elongate member 110 by locking to it. The grippers 116 are arranged to receive the elongate member 110, and the proximal end 112 of the elongate member 110 is passed through the grippers 116. As shown in FIG. 6A, the second gripper 118 can be moved proximally. From the perspective of the second gripper 118, proximally moving the second gripper 118 along the elongate member 110 is equivalent to distally moving the elongate member along the second gripper 118. Accordingly, the second gripper 118 can lock to the elongate member 110 and carry the elongate member 110 through the duration of the second gripper's 118 proximal movement. Because the distal end 116 of the elongate member 110 is fixed, the proximal movement of the elongate member 110 with the second gripper 118 encourages tension in the elongate member 110. As shown in 6B, upon reaching the conclusion of its proximate travel, the second gripper 118 can move distally along the elongate member 110, thereby releasing its grip on the elongate member 110. Although the tension in the elongate member 110 would tend to shift the elongate member 110 distally, the first gripper 20 remains substantially fixed and locks to the elongate member 110, thereby preventing distal movement and maintaining the tension. As shown in FIGS. 6C and 6D, the above-described steps may be repeated.

Thus, an improved tensioning device and method have been provided as described above. While the structure has been described in terms of certain specific embodiments, there is no intention to limit the invention to the same. It will be understood that the foregoing is only illustrative of the principles of the invention, and that various modifications, alterations, and combinations can be made by those skilled in the art without departing from the scope and spirit of the invention. Accordingly, it is not intended that the invention be limited, except as by the appended claims.

What is claimed is:

1. A elongate member tensioning device comprising:
a body with a distal end and a proximal end,
a tensioning feature comprising:
a trigger linkedly connected to at least one of a first gripper and a second gripper, and
the second gripper being in communication with a spring coupled to an indicator;
a handle; and
a rotating feature comprising:
an offset rod, a rotatable shaft with a lumen at the distal end of the body, a rotatable crank at the proximal end of the body,
a first set of gears in communication with the crank and the offset rod, and a second set of gears in communication with the shaft and the offset rod.

2. The device of claim 1, wherein the rotating feature operates independently of the tensioning feature.

3. The device of claim 1, wherein the trigger is linkedly connected to the first gripper and the second gripper.

4. The device of claim 1, wherein moving the trigger proximally moves the second gripper distally.

5. The device of claim 1, further comprising a mechanism that releases the second gripper's grip on the elongate member.

6. The device of claim 1, wherein the distal end of the shaft is configured to interface a compressible implant.

7. The device of claim 1, wherein the first and second grippers are aligned with the shaft lumen so as to simultaneously receive an elongate member.

8. A tensioning device comprising:
a body housing a first gripper and a second gripper,
the first and second grippers aligned with the shaft lumen so as to simultaneously receive an elongate member, and
a trigger linked to the body to and to at least one gripper.

9. The device of claim 8, wherein the crank and rotatable shaft operate independent of the first and second grippers.

10. The device of claim 8, further comprising a mechanism that releases the second gripper's grip on the elongate member.

11. The device of claim 8, further comprising a crank coupled to the body and in communication with a rotatable shaft with a lumen.

12. A method for tensioning an elongate member comprising:
providing a first gripper and a second gripper,
configuring the first and second grippers to receive an elongate member having a proximal end and a fixed distal end,
passing the proximal end of the elongate member through the first and second grippers,
proximally moving the second gripper along the elongate member, the second gripper thereby gripping and encouraging tension in the elongate member, and
distally moving the second gripper along the elongate member, the second gripper thereby loosening its grip on the elongate member, but the first gripper thereby maintaining tension in the elongate member.

13. The method of claim 12, further comprising repeating, as necessary, the steps of proximally and distally moving the second gripper to achieve the desired level of tension.
